(19) United States
(12) Patent Application Publication
(10) Pub. No.: US 2013/0131558 A1
(43) Pub. Date: May 23, 2013

(54) METHOD AND APPARATUS FOR PREVENTING LOCALIZED STASIS OF CEREBROSPINAL FLUID

(71) Applicant: Susan J. Lee, Studio City, CA (US)

(72) Inventor: Susan J. Lee, Studio City, CA (US)

(21) Appl. No.: 13/300,191

(22) Filed: Nov. 18, 2011

(51) Int. Cl. A61N 7/00; A61H 1/00

(52) U.S. Cl. 601/2; 601/46

(57) ABSTRACT

A system and method for treating, inhibiting or ameliorating Alzheimer's disease in a patient, comprising one or more transducers operatively connected to a power source for radiating vibrational or acoustical energy to the maxilla of the patient, wherein the energy is sufficient to radiate through the paranasal sinuses of the patient to the base of the patient's skull to assist in cerebrospinal fluid clearance and inhibit localized stasis of cerebrospinal fluid in the brain.

### CROSS-REFERENCE TO RELATED APPLICATIONS

This application claims the benefit of the filing date of U.S. Non-Provisional application Ser. No. 12/037,584, filed Feb. 26, 2008, the contents of which are incorporated herein by reference.

### FIELD OF THE INVENTION

This application relates generally to the prevention and treatment of dementia of the Alzheimer's type, and more particularly, to a method, system and apparatus for preventing localized stasis of cerebrospinal fluid.

### BACKGROUND OF THE INVENTION

It has been over 100 years since the first diagnosis of Alzheimer's Disease (“AD”), yet there is still no clear understanding of the disease process. The Alzheimer's Association recently projected that 5.1 million Americans are stricken with the disease and someone is newly diagnosed every 72 seconds in the United States. AD manifests itself with progressively impaired memory leading to mental confusion or dementia as the disease systematically kills off nerve cells in the brain. The staggering anticipated figures for what is to come in the future with the aging baby boomer population demands that progress be made in preventative, diagnostic and therapeutic AD research.

Cerebrospinal fluid (“CSF”) stagnation and/or failure secondary to senescence has been implicated to have a fundamental role in AD dementia. CSF is the protective fluid that fills the empty spaces around the brain and spinal cord. CSF has a crucial homeostatic circulatory role for metabolites, proteins and ions in and out of the brain. Normal CSF volume in an adult is about 140 milliliters and the entire volume turns over 3 or 4 times during the course of one day. It is known that CSF production and turnover decreases with age. CSF stagnation and/or failure will result in disruption of this tightly regulated equilibrium of fluid.

CSF has to flow through a labyrinth of tightly coiled sulci and fissures deep within the brain, sometimes against gravity, in key limiting anatomic locations. However, the brain lacks any local mechanical system, such as valves or motile appendages (e.g., cilia) to promote flow in a certain direction. Instead, CSF flow and velocity are driven by the cardiac cycle and modulated by venous pressure changes during respiration. Thus, it is feasible to have conditions under which chronic, localized stasis of CSF would lead to an accumulation of toxins and metabolites, which serves as a chronic, low-grade, physiological insult that cascades into the slow and insidious pathogenesis of AD as one ages.

To combat CSF stagnation, ventriculoperitoneal shunts, such as Eunoe, Inc.'s COGNIShunt® system, have been surgically implanted in the brains of AD afflicted patients as an artificial means to promote better CSF flow and turnover. While the results of this surgical procedure have been promising with respect to hindering the progression of dementia, the AD patient must undergo an extremely invasive surgical procedure in which a foreign body (i.e., ventriculoperitoneal shunt and its associated drainage tubing) is introduced into the patient's body. In addition, the potential benefits of this procedure are limited since the ventriculoperitoneal shunt may promote better CSF flow at a global level, but does not address or target specific, limiting anatomic areas of the brain that are most susceptible to CSF stasis.

Accordingly, there is a need for a non-invasive procedure that will prevent or reduce CSF stasis in the brain. There is also a need for a non-invasive procedure that promotes better CSF flow and turnover at specific, limiting anatomic areas of the brain that are most susceptible to CSF stasis.

### SUMMARY OF THE INVENTION

A system, apparatus and method for treating, inhibiting or ameliorating Alzheimer's disease in a patient, comprising one or more transducers operatively connected to a power source for radiating vibrational or acoustical energy to the maxilla of the patient, wherein the energy is sufficient to radiate through the paranasal sinuses of the patient to the base of the patient's skull to assist in cerebrospinal fluid clearance and inhibit localized stasis of cerebrospinal fluid in the brain.

These and other advantages of the present disclosure will be apparent to those of ordinary skill in the art by reference to the following detailed description and the accompanying drawings.

### BRIEF DESCRIPTION OF THE DRAWINGS

FIG. 1 illustrates an exemplary embodiment of a CSF stasis prevention system and apparatus in accordance with the present disclosure;

FIG. 2 is a schematic illustrating the power generator for one or more driving transducers in the CSF stasis prevention system and apparatus illustrated in FIG. 1;

FIG. 3 illustrates a second exemplary embodiment of the CSF stasis prevention system and apparatus in accordance with the present disclosure;

FIG. 4 illustrates a third exemplary embodiment of the CSF stasis prevention system and apparatus in accordance with the present disclosure;

FIG. 5 illustrates a fourth exemplary embodiment of the CSF stasis prevention system and apparatus in accordance with the present disclosure; and

FIG. 6 illustrates a fifth exemplary embodiment of the CSF stasis prevention system and apparatus in accordance with the present disclosure.

### DETAILED DESCRIPTION

Methods, apparatuses and systems for preventing CSF stasis in the brain are disclosed herein. The CSF stasis prevention methods, apparatuses and systems transfer vibrational or acoustical energy via bone conduction and/or air conduction through the paranasal sinuses to the base of the skull preferably using intraoral and/or extraoral/external devices.

The paranasal sinuses are thin-walled, mucosa-lined, hollow, air-filled spaces or cavities within the craniomaxillofacial complex (i.e., the bones of the face and skull) that communicate with the nasal cavity. Humans have four paired paranasal sinuses, which are often referred to according to the bone in which the particular sinus lies. These are the maxillary, ethmoid, frontal and sphenoid sinuses. The maxillary sinuses lie in the maxillary (cheek) bones under the eyes. The ethmoid sinuses lie in the ethmoid bone between the nose and eyes. The frontal sinuses lie in the frontal bone that forms part of the forehead above the eyes. The sphenoid sinuses lie in the sphenoid bone at the center of the skull base under the pituitary gland.

The biological function of the paranasal sinuses has been the subject of much debate and research, but, to date, there has been no widely accepted or, for that matter, discernible function associated with paranasal sinuses.

The CSF stasis prevention apparatuses, systems and methods disclosed herein utilize the paranasal sinuses to transmit vibrational or acoustical energy via bone and/or air conduction to the forehead and base of the skull. It is believed that the paranasal sinuses are hollow, resonating structures that naturally transmit vibrational or acoustical energy to the base of the skull and forehead during activities such as chewing, singing or being exposed to loud music/noise. This energy or resonance affects CSF dynamics by creating fluid waves in the CSF to prevent localized stasis in those deeper tightly coiled parts of the brain where CSF may have to flow against gravity.

This is particularly so with respect to the sphenoid sinuses, which are strategically located anterior, medial and inferior to the hippocampus. The hippocampus is a part of the brain located in the medial temporal lobe that plays a part in long-term memory and is most critically involved in AD as it is one of the first regions of the brain to suffer damage.

As will be described in greater detail below, intraoral and/or external devices are disclosed herein for use by a patient to transmit vibrational or acoustical energy through the paranasal sinuses—in particular, the sphenoid sinuses—to the base of the skull and forehead. The intraoral and/or external devices include one or more transducers connected to an energy source such that vibrational or acoustical energy is applied to the maxilla, preferably at the base of the midface. The vibrational or acoustical energy may be at infrasonic, sonic, supersonic or ultrasonic frequencies. In this way, the energy will travel via bone and/or air conduction from the maxilla to the paranasal sinuses and ultimately to the base of the skull, where tightly coiled structures of the brain, such as the hippocampus lying posterior and lateral to the sphenoid sinuses, are assisted in CSF clearance and prevent localized CSF stasis.

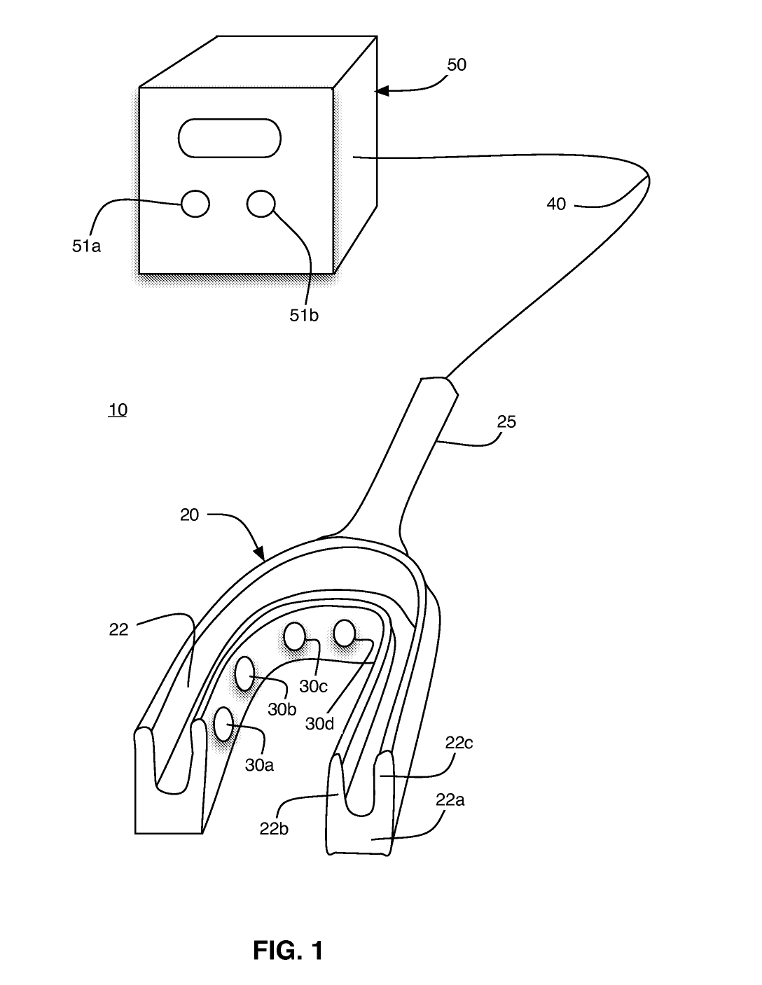

An exemplary, preferred embodiment of a CSF stasis prevention system 10 is illustrated in FIG. 1. The CSF stasis prevention system 10 includes a mouthpiece 20 capable of snuggly fitting over a patient's upper teeth. Alternatively, the mouthpiece 20 can be configured to fit over the upper jaw for patients without teeth. The mouthpiece 20 may be made of any suitable material that is biocompatible and safe for use within the mouth of a patient, such as polymethylmethacrylate (“PMMA”), acrylic, or other suitable plastic or thermoplastic materials. The mouthpiece 20 can be custom made to snugly, but comfortably fit over the upper teeth or upper jaw of the particular patient.

The mouthpiece 20 preferably includes a generally U-shaped groove 22 for enclosing the patient's teeth or upper jaw. The groove 22 is defined by a base 22a and two upstanding walls 22b, 22c. One or more transducers 30a, 30b, 30c, 30d are mounted in a conventional manner on the interior side of upstanding wall 22b. The central portion of the mouthpiece 20 in which the transducers 30a, 30b, 30c, 30d are located may optionally be enclosed or sealed to encapsulate the transducers.

The transducers 30a, 30b, 30c, 30d may be any type of transducer suitable for converting electrical energy into mechanical/vibrational energy, such as a resonance-type ultrasonic, sonic or supersonic transducer. These transducers produce high intensity waves or energy by, for example, applying the output of an electronic oscillator to a thin wafer of piezoelectric material (e.g., lead zirconate-titanate (PdZrTi or PZT) or barium titanate (BaTiO3)), which acts as a vibration element. The transducers 30a, 30b, 30c, 30d and their respective wiring or leads are preferably coated or covered with a suitable electrical insulation material that is safe for use within the mouth of a patient, such as, for example, Teflon. The mouthpiece 20 preferably includes a hollow, elongated handle 25 through which one or more insulated electrical wires 40 pass. The insulated electrical wires 40, which may be bundled in the form of an insulated cord or cable, electrically connect the transducers 30a, 30b, 30c, 30d to an external power generator 50 that drives the transducers.

The external power generator 50 may be powered by any conventional power source, such as, for example, conventional AC power, battery or solar operated, or 110 volt or 220 volt electric power. The external power generator 50 supplies power and provides signals for controlling the transducers 30a, 30b, 30c, 30d. The output of the power generator 50 is preferably in the range of approximately 10 kilohertz to approximately 300 megahertz or more. In the preferred embodiment, the output of the power generator 50 can be adjusted via a frequency adjustment knob 51a connected to, for example, a variable controlled oscillator, to attain the optimal target frequency for the particular patient and the time of treatment can be selected via the timer 51b, after which time the power to the transducers 30a, 30b, 30c, 30d is disconnected or otherwise ceases.

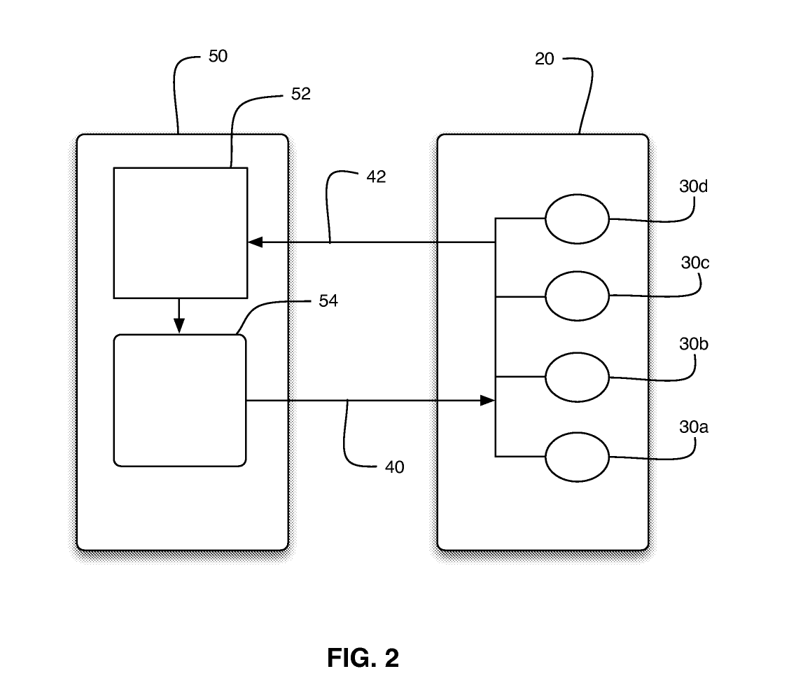

Referring to FIG. 2, the external power generator 50 preferably includes a processor or microprocessor 52 to generate control signals that are amplified by an output driver or amplifier 54 to the desired power level and imparted via wiring or cabling 40 on the transducers 30a, 30b, 30c, 30d. The processor 52 may dynamically tune the transducers 30a, 30b, 30c, 30d to the targeted frequency based on a feedback signal 42 received from the transducers.

The operation of the CSF stasis prevention system 10 is described below. The mouthpiece 20 is inserted into the patient's mouth so that the patient's upper teeth or upper jaw fit snugly in the grove 22. The CSF stasis prevention system 10 is either preprogrammed to the desired target frequency and length of time of treatment, or a practitioner selects the desired target frequency for the patient using knob 51a and the length of time of treatment using knob 51b. The power generator 50 supplies the desired output power to the transducers 30a, 30b, 30c, 30d, which radiate vibrational and/or acoustical energy at the desired frequency via bone and/or air conduction from the maxilla to the paranasal sinuses and ultimately to the base of the skull, where tightly coiled structures of the brain, such as the hippocampus lying posterior and lateral to the sphenoid sinuses, are assisted in CSF clearance and prevent localized CSF stasis.

While the CSF stasis prevention system 10 is described and illustrated as including a plurality of transducers 30a, 30b, 30c, 30d, it is understood that the system 10 requires a minimum of one transducer and may be utilized with any number of transducers that will produce the desired vibrational energy to be transmitted to the patient.

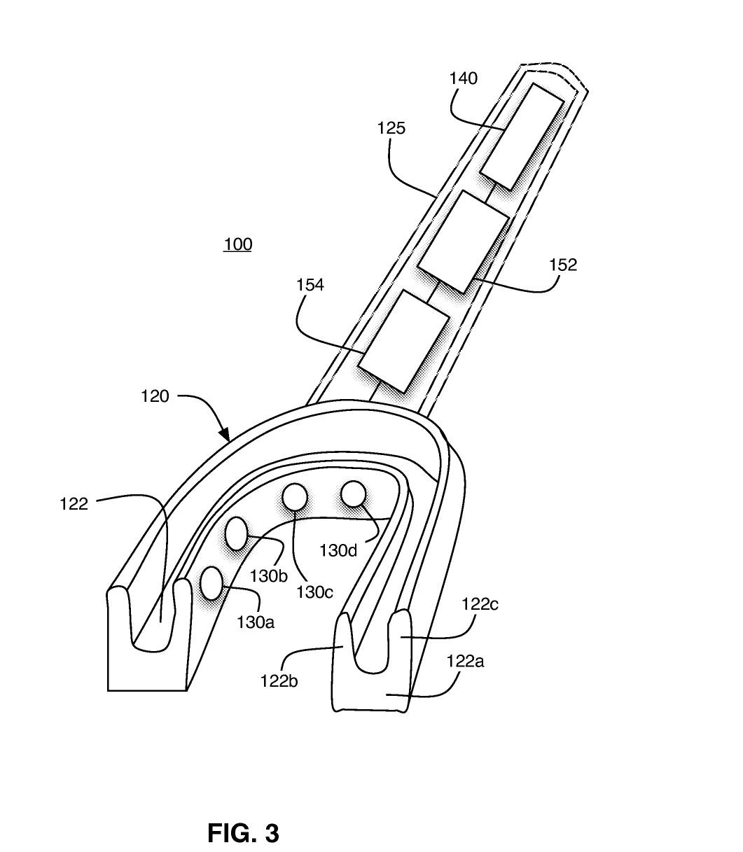

Another embodiment of the CSF stasis prevention system 100 is illustrated in FIG. 3. The CSF stasis prevention system 100 is similar to that illustrated in FIG. 1, but the CSF stasis prevention system 100 preferably has the logic and circuitry of the external power generator 50 built into the hollow, elongated handle 125.

The CSF stasis prevention system 100 preferably includes a mouthpiece 120, generally U-shaped channel 122, base 122a, upstanding walls 122b, 122c, and one or more transducers 130a, 130b, 130c, 130d similar to the mouthpiece 20, generally U-shaped channel 22, base 22a, upstanding walls 22b, 22c, and one or more transducers 30a, 30b, 30c, 30d of the above-described CSF stasis prevention system 10.

The handle 125 is preferably constructed of a rigid or semi-rigid material. One or more batteries 140, preferably rechargeable batteries, are located within the handle 125. A processor or microprocessor 152 is electrically connected to the battery(s) 140 to generate control signals that are amplified by an output driver or amplifier 154 to the desired power level and imparted on the transducers 130a, 130b, 130c, 130d. The processor 152 may dynamically tune the transducers 130a, 130b, 130c, 130d to the targeted frequency based on a feedback signal received from the transducers. Alternatively, the device may include a variable-controlled oscillator for adjusting and/or regulating power level being imparted on the transducers 130a, 130b, 130c, 130d.

The CSF stasis prevention system 100 is operated in a manner similar to that described above with respect to the CSF stasis prevention system 10. In addition, while the CSF stasis prevention system 100 is described and illustrated as including a plurality of transducers 130a, 130b, 130c, 130d, it is understood that the system 100 requires a minimum of one transducer and may be utilized with any number of transducers that will produce the desired vibrational energy to be transmitted to the patient.

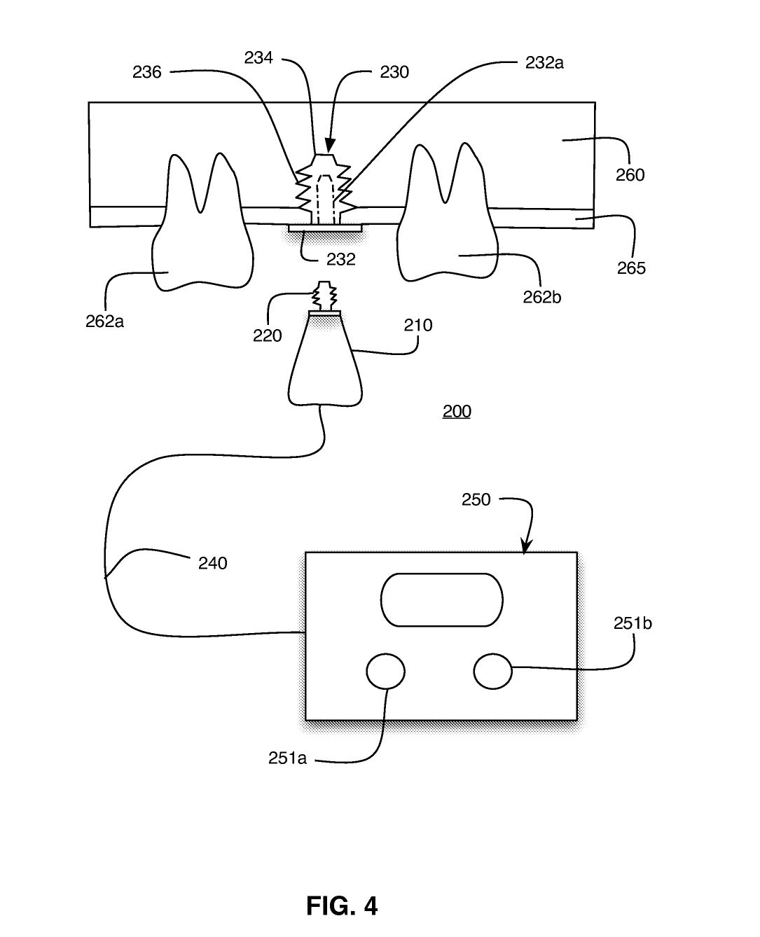

Another embodiment of the CSF stasis prevention system 200 is illustrated in FIG. 4. This embodiment may be particularly advantageous for patients who are edentulous. The CSF stasis prevention system 200 preferably includes an implant 230 that is surgically inserted into the maxillary bone or upper jaw 260 below the gingival tissue 265. The implant 230 preferably includes a head portion 232, a distal end 234, and a threaded portion 236 between the head portion and distal end. The threaded portion 236 may have a specific surface, including, but not limited to, an acid-etched or sand blasted surface, to facilitate osseointegration with the bone 260 and may include a self-tapping region with incremental cutting edges that allow the implant 230 to be inserted into the bone 260 without the need for a bone tap. The implant 230 may, for example, be made of titanium, tantalum, cobalt, chromium, stainless steel, or alloys thereof.

The head portion 232 of the implant 230 preferably includes a threaded cavity 232a that will facilitate attachment of a transducer 210 to the implant 230. The transducer 210 may be any type of transducer suitable for converting electrical energy into vibrational and/or acoustical energy, such as a resonance-type ultrasonic, sonic or supersonic transducer. These transducers produce high intensity waves or energy by, for example, applying the output of an electronic oscillator to a thin wafer of piezoelectric material (e.g., lead zirconate-titanate (PdZrTi or PZT) or barium titanate (BaTiO3)), which acts as a vibration element.

The transducer 210 preferably includes a threaded portion 220 at one end thereof that is received within and releasably engages corresponding threads formed within the cavity 232a of the implant 230. Alternatively, the transducer 210 may have a threaded cavity that releasably engages a threaded post or abutment extending from the head portion 232 of the implant 230. Other conventional techniques may be used to releasably attach the transducer 210 to the implant 230. For example, a conventional abutment or post may be attached to or integral with the implant 230, and the transducer 210 may be releasably attached to the abutment or post extending from the implant 230.

The transducer 210 is electrically connected to an external power generator 250 via an insulated wire or cable 240. The external power generator 250 supplies power and provides signals for controlling one or more transducers 210. The external power generator 250 may be powered by any conventional power source, such as, for example, conventional AC power, battery or solar operated, or 110 volt or 220 volt electric power. The output of the power generator 250 is preferably in the range of approximately 10 kilohertz to approximately 300 megahertz or more. In the preferred embodiment, the output of the power generator 250 can be adjusted via a frequency adjustment knob 251a connected to, for example, a variable controlled oscillator, to attain the optimal target frequency for the particular patient and the time of treatment can be selected via the timer 251b, after which time the power to the transducer 210 is disconnected or otherwise ceases.

Like the embodiment illustrated in FIG. 2, the external power generator 250 preferably includes a processor or microprocessor to generate control signals that are amplified by an output driver or amplifier to the desired power level and imparted via wiring or cabling 240 on the transducer 210. The processor may dynamically tune the transducer 210 to the targeted frequency based on a feedback signal received from the transducer.

The operation of the CSF stasis prevention system 200 is described below. After the implant 230 is implanted into the maxillary bone or jawbone 260 of the patient, the transducer 210 may be releasably connected to the implant 230 by, for example, inserting the threaded end 220 of the transducer 210 into the threaded cavity 232a formed in the implant. The CSF stasis prevention system 200 may be preprogrammed with the desired target frequency and the length of time of treatment, or a practitioner may select or adjust the desired target frequency for the patient using knob 251a and the length of time of treatment using knob 251b. The power generator 250 supplies the desired output power to the transducer 210, which radiates vibrational energy at the desired frequency through the implant 230 and into the maxilla bone 260. This vibration energy radiates via bone and/or air conduction from the maxilla to the paranasal sinuses and ultimately to the base of the skull, where tightly coiled structures of the brain, such as the hippocampus lying posterior and lateral to the sphenoid sinuses, are assisted in CSF clearance and prevent localized CSF stasis.

While the CSF stasis prevention system 200 is described above as including a single transducer 210, it is understood that the transducer 210 may be comprised of multiple transducers that are stacked or otherwise incorporated therein.

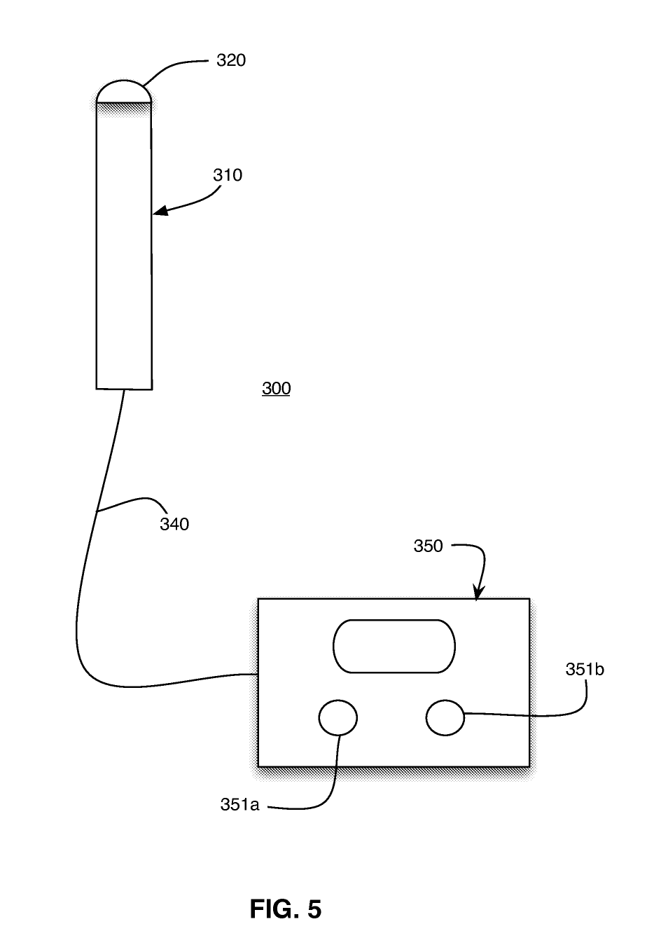

Another embodiment of the CSF stasis prevention system 300 is illustrated in FIG. 5. The CSF stasis prevention system 300 includes a wand 310 having a transducer 320 located at or near one end of the wand. The wand 310 is preferably made of a rigid or semi-rigid, biocompatible material such as, for example, plastic or thermoplastic.

The transducer 320 may be either mounted at the end of the wand 310 or within a cavity (not shown) in the wand 310. While a single transducer 320 is illustrated in FIG. 5, it is understood that the transducer 320 may be comprised of multiple transducers that are stacked or otherwise incorporated on or within the wand 310. The transducer 320 may be any type of transducer suitable for converting electrical energy into vibrational and/or acoustical energy, such as a resonance-type ultrasonic, sonic or supersonic transducer. These transducers produce high intensity waves or energy by, for example, applying the output of an electronic oscillator to a thin wafer of piezoelectric material (e.g., lead zirconate-titanate (PdZrTi or PZT) or barium titanate (BaTiO3)), which acts as a vibration element.

If the transducer 320 is mounted at the end of the wand 310, it should be preferably coated with a protective, biocompatible coating, such as, for example, Teflon, that seals and electrically insulates the transducer, and provides a smooth surface for applying the transducer to a patient's teeth, gums or other tissue. Alternatively, if the transducer 320 is located within a cavity in the wand 310, then the end of the wand 310 where the transducer 320 is located should preferably be smooth to provide a comfortable surface for applying the end of the wand to a patient's teeth, gums or other tissue. The end of the wand may also be provided with protective, biocompatible surface or coating to provide a comfortable surface for applying the end of the wand to a patient's teeth, gums or other tissue.

The transducer 320 is electrically connected to an external power generator 350 via an insulated wire or cable 340. The external power generator 350 supplies power and provides signals for controlling the transducer 320. The external power generator 350 may be powered by any conventional power source, such as, for example, conventional AC power, battery or solar operated, or 110 volt or 220 volt electric power. The output of the power generator 350 is preferably in the range of approximately 10 kilohertz to approximately 300 megahertz or more. In the preferred embodiment, the output of the power generator 350 can be adjusted via a frequency adjustment knob 351a connected to, for example, a variable controlled oscillator, to attain the optimal target frequency for the particular patient and the time of treatment can be selected via the timer 351b, after which time the power to the transducer 320 is disconnected or otherwise ceases.

Like the embodiment illustrated in FIG. 2, the external power generator 350 preferably includes a processor or microprocessor to generate control signals that are amplified by an output driver or amplifier to the desired power level and imparted via wiring or cabling 340 on the transducer 320. The processor may dynamically tune the transducer 320 to the targeted frequency based on a feedback signal received from the transducer.

The operation of the CSF stasis prevention system 300 is described below. The CSF stasis prevention system 300 may be preprogrammed with the desired target frequency and the length of time of treatment, or a practitioner may select or adjust the desired target frequency for the patient using knob 351a and the length of time of treatment using knob 351b. The power generator 350 supplies the desired output power to the transducer 320, which radiates vibrational energy at the desired frequency. The end of the wand 310 where the transducer 320 is located is preferably placed intraorally against the patient's upper teeth or gums (or externally against skin on the patient's face in proximity to the upper teeth, gums or cheekbone) and the vibrational and/or acoustical energy from the transducer 320 radiates via bone and/or air conduction from the maxilla to the paranasal sinuses and ultimately to the base of the skull, where tightly coiled structures of the brain, such as the hippocampus lying posterior and lateral to the sphenoid sinuses, are assisted in CSF clearance and prevent localized CSF stasis.

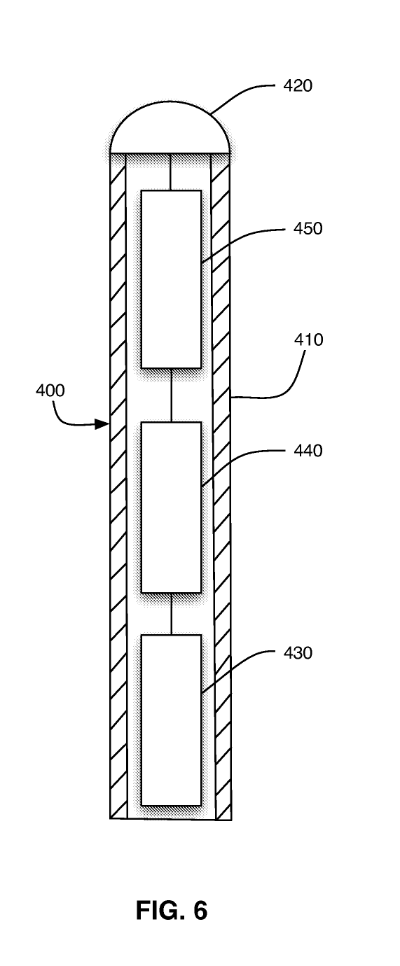

Another embodiment of the CSF stasis prevention system 400 is illustrated in FIG. 6. The CSF stasis prevention system 400 is similar to that illustrated in FIG. 5, but the CSF stasis prevention system 400 preferably has the logic and circuitry of the external power generator 350 built into the wand 410. The wand 410 is preferably hollow and has one or more transducers 420 mounted on or near the end of the wand 410. The transducer 420 is similar to the transducer 320 described in the preceding embodiment.

As with the wand 310 illustrated in FIG. 5, the transducer 420 may be coated with a protective, biocompatible coating, such as, for example, Teflon, that seals and electrically insulates the transducer, and provides a smooth surface for applying the transducer to a patient's teeth, gums or other tissue. Alternatively, if the transducer 420 is located inside the wand 410, then the end of the wand where the transducer 420 is located should preferably be smooth to provide a comfortable surface for applying the end of the wand to a patient's teeth, gums or other tissue. The end of the wand may also be provided with protective, biocompatible surface or coating to provide a comfortable surface for applying the end of the wand to a patient's teeth, gums or other tissue.

One or more batteries 430, preferably rechargeable batteries, are located within the wand 410. A processor or microprocessor 440 is electrically connected to the battery(s) 430 to generate control signals that are amplified by an output driver or amplifier 450 to the desired power level and imparted on the transducer 420. The processor 440 may dynamically tune the transducer 420 to the targeted frequency based on a feedback signal received from the transducer. Alternatively, the device 400 may include a variable-controlled oscillator for adjusting and/or regulating power level being imparted on the transducer 420.

The CSF stasis prevention system 400 is operated in a manner similar to that described above with respect to the CSF stasis prevention system 300 illustrated in FIG. 5.

The above-described intraoral and external devices are exemplary, preferred embodiments for generating and transferring vibrational and/or acoustical energy via bone and/or air conduction from the maxilla to the paranasal sinuses and ultimately to the base of the skull, where tightly coiled structures of the brain, such as the hippocampus lying posterior and lateral to the sphenoid sinuses, are assisted in CSF clearance and prevent localized CSF stasis. These devices will aid in preventing localized stasis of CSF in key, anatomic locations of the brain and may serve as a preventative measure against AD and/or slow its progression.

The above-described intraoral and external devices may be utilized on a patient that is in a reclined or supine position to further assist CSF clearance and prevent localized CSF stasis.

It is understood that other devices radiating acoustical energy can also be utilized to transfer energy via bone conduction to the base of the skull and prevent localized stasis of CSF in key, anatomic locations of the brain and may serve as a preventative measure against AD and/or slow its progression. For example, acoustical energy generated by a speaker or other type of transducer emitting acoustical energy may be utilized to achieve these results.

In addition, the devices disclosed herein may also aid in distributing and dispersing intrathecal and intravenous medications, compounds, transplanted cells and tissues, and genetically engineered cells within CSF. Similarly, the devices disclosed herein may aid in clinical and scientific research involving the transport of molecules, ions and proteins in and out of CSF.

Having described and illustrated the principles of this application by reference to one or more preferred embodiments, it should be apparent that the preferred embodiment(s) may be modified in arrangement and detail without departing from the principles disclosed herein and that it is intended that the application be construed as including all such modifications and variations insofar as they come within the spirit and scope of the subject matter disclosed herein.

I claim:

1. A method for treating, inhibiting or ameliorating localized stasis of cerebrospinal fluid in the brain of a patient, comprising administering vibrational or acoustical energy to the maxilla of the patient, wherein the energy is sufficient to radiate through the paranasal sinuses of the patient to the base of the patient's skull to assist in cerebrospinal fluid clearance and inhibit localized stasis of cerebrospinal fluid in the brain.

2. The method according to claim 1, wherein the vibrational or acoustical energy is administered to the maxilla via one or more transducers operatively connected to a power source.

3. The method according to claim 2, wherein the one or more transducers radiate energy to one or more upper teeth of the patient.

4. The method according to claim 2, further comprising inserting a mouthpiece over the patient's upper teeth or upper jaw, wherein the one or more transducers are disposed on the mouthpiece and direct vibrational or acoustical energy to the patient's upper teeth or upper jaw.

5. The method according to claim 2, wherein the one or more transducers are ultrasonic transducers.

6. The method according to claim 2, wherein the one or more transducers are sonic transducers.

7. The method according to claim 2, wherein the one or more transducers are supersonic transducers.

8. The method according to claim 2, wherein the transducer is releasably connected to an implant disposed in the maxilla of the patient.

9. The method according to claim 2, further comprising causing a wand to contact the patient's upper teeth, gums or tissue, wherein the one or more transducers are disposed on a wand and direct vibrational or acoustical energy to the patient's upper teeth, gums or tissue.

10. A method for treating, inhibiting or ameliorating Alzheimer's disease in a patient, comprising administering vibrational or acoustical energy to the maxilla of the patient, wherein the energy is sufficient to radiate through the paranasal sinuses of the patient to the base of the patient's skull to assist in cerebrospinal fluid clearance and inhibit localized stasis of cerebrospinal fluid in the brain.

11. The method according to claim 10, wherein the vibrational or acoustical energy is administered to the maxilla via one or more transducers operatively connected to a power source.

12. The method according to claim 11, wherein the one or more transducers radiate energy to one or more upper teeth of the patient.

13. The method according to claim 11, further comprising inserting a mouthpiece over the patient's upper teeth or upper jaw, wherein the one or more transducers are disposed on the mouthpiece and direct vibrational or acoustical energy to the patient's upper teeth or upper jaw.

14. The method according to claim 11, wherein the one or more transducers are ultrasonic transducers.

15. The method according to claim 11, wherein the one or more transducers are sonic transducers.

16. The method according to claim 11, wherein the one or more transducers are supersonic transducers.

17. The method according to claim 11, wherein the transducer is releasably connected to an implant disposed in the maxilla of the patient.

18. The method according to claim 11, further comprising causing a wand to contact the patient's upper teeth, gums or tissue, wherein the one or more transducers are disposed on a wand and direct vibrational or acoustical energy to the patient's upper teeth, gums or tissue.

19. A system for treating, inhibiting or ameliorating localized stasis of cerebrospinal fluid in the brain of a patient, comprising one or more transducers operatively connected to a power source for radiating vibrational or acoustical energy to the maxilla of the patient, wherein the energy is sufficient to radiate through the paranasal sinuses of the patient to the base of the patient's skull to assist in cerebrospinal fluid clearance and inhibit localized stasis of cerebrospinal fluid in the brain.

20. The system according to claim 19, further comprising a power generator operatively connected and supplying power to the one or more transducers at a desired frequency.
